(19) United States
(12) Patent Application Publication
(10) Pub. No.: US 2022/0339325 A1
(43) Pub. Date: Oct. 27, 2022

(54) CORROSION-RESISTANT AND LEAK-PROOF STOMA PASTE WITH SELF-REGULATION OF STRENGTH AND PREPARATION METHOD

(71) Applicant: Zhende Medical Co., Ltd., Shaoxing (CN)

(72) Inventors: Lanqi FENG, Shaoxing City (CN); Jianguo LU, Shaoxing (CN)

(73) Assignee: Zhende Medical Co., Ltd., Shaoxing (CN)

(21) Appl. No.: 17/664,600

(22) Filed: May 23, 2022

Related U.S. Application Data
(63) Continuation of application No. PCT/CN2021/109804, filed on Jul. 30, 2021.

(30) Foreign Application Priority Data
Apr. 27, 2021 (CN) 202110459156.6

(51) Int. Cl. A61L 28/00; A61K 36/30; A61K 36/232; A61K 36/185

(52) U.S. Cl. CPC A61L 28/0038; A61K 36/30; A61K 36/232; A61K 36/185; A61L 28/0026; A61L 28/0003; A61L 2400/14

(57) ABSTRACT

A corrosion-resistant and leak-proof stoma paste with self-regulation of strength is provided in this disclosure, with a formula comprising 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight. A product of the present disclosure can self-regulate hardness of the paste according to amount of stoma excreta through cooperation of the iota-carrageenan and the sericite to improve corrosion resistance of the paste; and synergistic effect provided by the iota-carrageenan and the sericite powder can improve strength and corrosion resistance of the paste.

### CROSS-REFERENCE TO RELATED APPLICATIONS

This application is a continuation of International Application No. PCT/CN2021/109804 filed Jul. 30, 2021, which claims the benefit of priority to Chinese Application No. 202110459156.6, filed Apr. 27, 2021, the benefit of priority of each of which is claimed herein and which applications are hereby incorporated by reference herein in their entirety.

### TECHNICAL FIELD

The disclosure relates to a field of medical materials, in particular to a corrosion-resistant and leak-proof stoma paste with self-regulation of strength and a preparation method thereof.

### BACKGROUND ART

Currently, best-selling leak-proof pastes in China are mainly divided into alcoholic ones and non-alcoholic ones. The alcoholic paste present good shaping ability and short curing time, but it contains isopropanol and other ingredients, which can irritate skin to a certain extent and is not conducive to use of dermatitis patients. The non-alcoholic paste has no irritation to skin with poor shaping ability, and it is easy to be squeezed out when it is used with a stoma rubber tray, which increases difficulty of stoma care. A non-alcoholic leak-proof paste for stoma care and preparation method thereof are disclosed in Chinese patent application title by “A non-alcoholic leak-proof paste for stoma care and preparation method thereof (with an application publication number: CN108744066A)”. A corrosion-resistant and leak-proof stoma paste and preparation method thereof are disclosed Chinese patent application document titled by “A corrosion-resistant and leak-proof stoma paste and preparation method thereof (with an application publication number: CN108992713A)”. Inorganic clay is introduced into these two types of leak-proof pastes to improve corrosion-resistant performance of the pastes, with low paste strength. A type of paste is required in the market that can solve problems of poor corrosion resistance and low paste strength of existing leak-proof pastes.

Currently, the leak-proof paste only plays a simple role in filling gaps around the stoma and can only prevent generation of skin complications, but does not have a function of treating the skin complications. A type of paste is required in the market that can prevent leakage and treat the skin complications at the same time.

The disclosure solves above two problems, and provides a leak-proof paste with good corrosion resistance and high paste strength, which can treat the skin complications at the same time.

### SUMMARY

In order to solve shortcomings of the prior art, the present disclosure aims to provide a corrosion-resistant and leak-proof stoma paste with self-regulation of strength and preparation method thereof. A product of the present disclosure can self-regulate hardness of the paste according to amount of stoma excreta to improve corrosion resistance of the paste, and introduce stoma skin complication medicine, which can prevent leakage and play a therapeutic effect on the skin complications of the stoma at the same time.

In order to achieve above objects, the disclosure employs the following technical scheme.

A corrosion-resistant and leak-proof stoma paste with self-regulation of strength is provided, with a formula comprising 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight.

For the corrosion-resistant and leak-proof stoma paste with self-regulation of strength, the formula further includes 5-15 parts of the stoma skin complication medicine by weight.

For the corrosion-resistant and leak-proof stoma paste with self-regulation of strength, the stoma skin complication medicine includes 1-3 parts of

Lithospermum Root, 1-3 parts of Angelica sinensis and 3-9 parts of sesame oil, by weight.

For the corrosion-resistant and leak-proof stoma paste with self-regulation of strength, a weight ratio of the Lithospermum Root to the sesame oil is 1: 3. For the corrosion-resistant and leak-proof stoma paste with self-regulation of strength, the liquid rubber with the molecular weight ranging from 2000 to 5000 is one or more of polyisobutylene, liquid polyisoprene and liquid styrene-butadiene rubber with a molecular weight ranging from 2000 to 5000.

For the corrosion-resistant and leak-proof stoma paste with self-regulation of strength, the mineral oil is one or more of naphthenic oil, white oil and paraffin oil.

For the corrosion-resistant and leak-proof stoma paste with self-regulation of strength, the plant polysaccharide is one or more of tamarind seed polysaccharide, seaweed polysaccharide, chitosan oligosaccharide, tremella polysaccharide, hawthorn polysaccharide and oat β- glucan.

A preparation method of a corrosion-resistant and leak-proof stoma paste with self-regulation of strength includes the following steps.

In Step 1, materials is prepared according to a formula.

The formula includes 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight.

In Step 2, the liquid rubber with the molecular weight ranging from 2000 to 5000, the mineral oil, the iota-carrageenan, the plant polysaccharide and the sericite are evenly mixed and stirred to obtain a mixed paste, which is packaged to obtain a finished product.

A preparation method of a corrosion-resistant and leak-proof stoma paste with self-regulation of strength includes the following steps. In Step 1, materials are prepared according to a formula.

The formula includes 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, 1-3 parts of Lithospermum Root, 1-3 parts of Angelica sinensis and 3-9 parts of sesame oil, by weight.

In Step 2, the sesame oil is heated to 90 ° C and then the weighed Lithospermum Root and the Angelica sinensis are added, decocted for lh, and filtered to obtain medicinal oil.

In Step 3, the obtained medicinal oil, the liquid rubber with the molecular weight ranging from 2000 to 5000, the mineral oil, the iota-carrageenan, the plant polysaccharide and the sericite are evenly mixed and stirred to obtain a mixed paste, which is packaged to obtain a finished product.

According to the preparation method, a double planetary stirrer is adopted for stirring, with a rotating speed of 80-180 r/min. The disclosure has advantages as follows.

In the disclosure, the iota-carrageenan is adopted, which can form a strong gel network structure with calcium ions in the stoma excreta. The gel network structure is elastic and insoluble in water and its strength increases with increase of the calcium ions in solution, so the paste can self-regulate its strength with amount of the stoma excreta.

In the disclosure, the sericite is adopted, which has unique volume expansion effect and a two-dimensional sheet configuration with a high aspect ratio, excellent reinforcement effect is exhibited in rubber products, so it can strengthen the paste jointly with the carrageenan. In the disclosure, it is found that the iota-carrageenan and the sericite are cooperated to present a synergistic effect on the strength and corrosion resistance of the paste, thus improve the strength of the paste and enhance the corrosion resistance.

The Lithospermum Root is adopted in the stoma skin complication medicine. The Lithospermum Root contains shikonin, which has good effect of eliminating putridity and engendering flesh. At the same time, it has been found in research that the Lithospermum Root can inhibit growth activity of Candida albicans, and protect and promote healing of wounds at the same time. The Angelica sinensis can promote blood circulation, nourish blood and promote granulation. The sesame oil has efficacy of detoxifying and promoting granulation. Combination of these three medicines presents remarkable astringent effect in treating skin diseases, so it can achieve astringent effect for skin complications and prevent sores.

### DETAILED DESCRIPTION

The disclosure will be described in the following in detail with reference to specific embodiments.

A corrosion-resistant and leak-proof stoma paste with self-regulation of strength is provided, with a formula comprising 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight. As an example, the liquid rubber with the molecular weight ranging from 2000 to 5000 is one or more of polyisobutylene, liquid polyisoprene and liquid styrene-butadiene rubber with a molecular weight ranging from 2000 to 5000. The mineral oil is one or more of naphthenic oil, white oil and paraffin oil. The plant polysaccharide is one or more of tamarind seed polysaccharide, seaweed polysaccharide, chitosan oligosaccharide, tremella polysaccharide, hawthorn polysaccharide and oat μ- glucan. It should be note that all of examples herein are not exhaustive, and those that can achieve corresponding functions and effects are inspired by the present disclosure and are within protection scope of the present disclosure.

In order to achieve astringent effect for skin complications and prevent sores, the formula further includes 5-15 parts of the stoma skin complication medicine by weight. The stoma skin complication medicine includes 1-3 parts of Lithospermum Root, 1-3 parts of Angelica sinensis and 3-9 parts of sesame oil, by weight. As a preferred alternative, a weight ratio of the Lithospermum Root to the sesame oil is 1: 3. The Lithospermum Root contains shikonin, which has good effect of eliminating putridity and engendering flesh. At the same time, it has been found in research that the Lithospermum Root can inhibit growth activity of Candida albicans, and protect and promote healing of wounds at the same time. The Angelica sinensis can promote blood circulation, nourish blood and promote granulation. The sesame oil has efficacy of detoxifying and promoting granulation. Combination of these three medicines presents remarkable astringent effect in treating skin diseases, so it can achieve astringent effect for skin complications and prevent sores.

As an embodiment, a preparation method of a leak-proof paste without skin complication medicine of a stoma includes following steps.

In Step 1, materials is prepared according to a formula. In Step 2, a liquid rubber with a molecular weight ranging from 2000 to 5000, mineral oil, iota-carrageenan, plant polysaccharide and sericite are evenly mixed and stirred to obtain a mixed paste, which is packaged to obtain a finished product.

As another embodiment, a preparation method of a leak-proof paste with skin complication medicine of a stoma includes following steps. In Step 1, materials is prepared according to a formula.

The formula includes 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, 1-3 parts of Lithospermum Root, 1-3 parts of Angelica sinensis and 3-9 parts of sesame oil, by weight.

In Step 2, the sesame oil is heated to 90 ° C. and then the weighed Lithospermum Root and the Angelica sinensis are added, decocted for lh, and filtered to obtain medicinal oil. In Step 3, the obtained medicinal oil, the liquid rubber with the molecular weight ranging from 2000 to 5000, the mineral oil, the iota-carrageenan, the plant polysaccharide and the sericite are evenly mixed, then put into and stirred in a double planetary stirrer with a rotating speed of 80-180 r/min, and a mixed paste is finally introduced into an automatic filling machine to finish filling. It should be noted that stirring and packaging are only for preferred embodiments, which is not limited and exhaustive. Whatever can realize functions of stirring and packaging is within the protection scope of the present disclosure.

Samples were obtained according to the formula and preparation method of following embodiments for Experiments 1-2.

### Embodiment 1

A novel corrosion-resistant and leak-proof stoma paste with self-regulation of strength for treating stoma skin complications according to an embodiment of the disclosure includes following raw materials in parts by weight: 34 g of liquid rubber with a molecular weight ranging from 2000 to 5000, 10 g of mineral oil, 15 g of iota-carrageenan, 15 g of plant polysaccharide, 20 g of sericite powder, 2 g of Lithospermum Root, 2 g of Angelica sinensis and 6 g of sesame oil.

The Lithospermum Root, the Angelica sinensis and the sesame oil are accurately weighed according to a formula; the sesame oil is heated to 90° C. and then the weighed Lithospermum Root and the Angelica sinensis are added, decocted for 1 h, and filtered to obtain medicinal oil; and the obtained medicinal oil, the liquid rubber with the molecular weight ranging from 2000 to 5000, the mineral oil, the iota-carrageenan, the plant polysaccharide and the sericite are evenly mixed in a mixed ratio, then put into and stirred in a double planetary stirrer, and then a mixed paste is introduced into an automatic filling machine to finish filling.

### Embodiment 2

A novel corrosion-resistant and leak-proof stoma paste with self-regulation of strength for treating stoma skin complications according to an embodiment of the disclosure includes following raw materials in g: 24 g of liquid rubber with a molecular weight ranging from 2000 to 5000, 12 g of mineral oil, 20 g of iota-carrageenan, 20 g of plant polysaccharide, 15g of sericite powder, 1 g of Lithospermum Root, 1 g of Angelica sinensis and 3 g of sesame oil.

The Lithospermum Root, the Angelica sinensis and the sesame oil are accurately weighed according to a formula; the sesame oil is heated to 90° C. and then the weighed Lithospermum Root and the Angelica sinensis are added, decocted for 1 h, and filtered to obtain medicinal oil; and the obtained medicinal oil, the liquid rubber with the molecular weight ranging from 2000 to 5000, the mineral oil, the iota-carrageenan, the plant polysaccharide and the sericite are evenly mixed in a mixed ratio, then put into and stirred in a double planetary stirrer, and then a mixed paste is introduced into an automatic filling machine to finish filling.

### Embodiment 3

A novel corrosion-resistant and leak-proof stoma paste with self-regulation of strength for treating stoma skin complications according to an embodiment of the disclosure includes following raw materials in g: 16 g of liquid rubber with a molecular weight ranging from 2000 to 5000, 15 g of mineral oil, 28 g of iota-carrageenan, 12 g of plant polysaccharide, 18 g of sericite powder, 3 g of Lithospermum Root, 3 g of Angelica sinensis and 9 g of sesame oil.

The Lithospermum Root, the Angelica sinensis and the sesame oil are accurately weighed according to a formula; the sesame oil is heated to 90° C. and then the weighed Lithospermum Root and the Angelica sinensis are added, decocted for 1 h, and filtered to obtain medicinal oil; and the obtained medicinal oil, the liquid rubber with the molecular weight ranging from 2000 to 5000, the mineral oil, the iota-carrageenan, the plant polysaccharide and the sericite are evenly mixed in a mixed ratio, then put into and stirred in a double planetary stirrer, and then a mixed paste is introduced into an automatic filling machine to finish filling.

### Comparative Embodiment 1

A formula is as follows: 24 g of liquid rubber with a molecular weight ranging from 2000 to 5000, 12 g of mineral oil, 20 g of plant polysaccharide, 15 g of sericite powder, 1 g of Lithospermum Root, 1 g of Angelica sinensis and 3 g of sesame oil.

The Lithospermum Root, the Angelica sinensis and the sesame oil are accurately weighed according to the formula; the sesame oil is heated to 90° C. and then the weighed Lithospermum Root and the Angelica sinensis are added, decocted for 1 h, and filtered to obtain medicinal oil; and the obtained medicinal oil, the liquid rubber with the molecular weight ranging from 2000 to 5000, the mineral oil, the plant polysaccharide and the sericite are evenly mixed in a mixed ratio, then put into and stirred in a double planetary stirrer, and then a mixed paste is introduced into an automatic filling machine to finish filling.

The formula and preparation steps are the same as those of Embodiment 2, except that iota-carrageenan is not added in the formula.

### Comparative Embodiment 2

A formula is as follows: 24 g of liquid rubber with a molecular weight ranging from 2000 to 5000, 12 g of mineral oil, 20 g of iota-carrageenan, 20 g of plant polysaccharide, 1 g of Lithospermum Root, 1 g of Angelica sinensis and 3 g of sesame oil.

The Lithospermum Root, the Angelica sinensis and the sesame oil are accurately weighed according to a formula; the sesame oil is heated to 90° C. and then the weighed Lithospermum Root and the Angelica sinensis are added, decocted for 1 h, and filtered to obtain medicinal oil; and the obtained medicinal oil, the liquid rubber with the molecular weight ranging from 2000 to 5000, the mineral oil, the iota-carrageenan and the plant polysaccharide are evenly mixed in a mixed ratio, then put into and stirred in a double planetary stirrer, and then a mixed paste is introduced into an automatic filling machine to finish filling.

The formula and preparation steps are the same as those of Embodiment 2, except that sericite powder is not added in the formula.

Comparative Embodiment 3

A formula is as follows: 24 g of liquid rubber with a molecular weight ranging from 2000 to 5000, 12 g of mineral oil, 20 g of iota-carrageenan, 20 g of plant polysaccharide, and 15 g of sericite powder.

The liquid rubber with the molecular weight ranging from 2000 to 5000, the mineral oil, the iota-carrageenan, the plant polysaccharide and the sericite are evenly mixed in a mixed ratio, then put into and stirred in a double planetary stirrer, and then a mixed paste is introduced into an automatic filling machine to finish filling.

The formula and preparation steps are the same as those of Embodiment 2, except that Lithospermum Root, Angelica sinensis and sesame oil are not added in the formula.

Comparative Embodiment 4

A formula is as follows: 24 g of liquid rubber with a molecular weight ranging from 2000 to 5000, 12g of mineral oil, 20 g of plant polysaccharide, lg of Lithospermum Root, 1 g of Angelica sinensis and 3g of sesame oil.

The Lithospermum Root, the Angelica sinensis and the sesame oil are accurately weighed according to the formula; the sesame oil is heated to 90° C. and then the weighed Lithospermum Root and the Angelica sinensis are added, decocted for 1h, and filtered to obtain medicinal oil; and the obtained medicinal oil, the liquid rubber with the molecular weight ranging from 2000 to 5000, the mineral oil and the plant polysaccharide are evenly mixed in a mixed ratio, then put into and stirred in a double planetary stirrer, and then a mixed paste is introduced into an automatic filling machine to finish filling.

The formula and preparation steps are the same as those of Embodiment 2, except that iota-carrageenan and sericite powder are not added in the formula.

Experiment 1. Sample material performance test

Paste products finally obtained in above Embodiments 1-3 and Comparative Embodiments 1-4 were tested respectively.

A certain amount (10g) of the paste is squeezed into a 100ml beaker, a total weight M1 of the paste and the beaker is accurately weighed; 50ml of artificial intestinal fluid is taken into the beaker containing the paste, the beaker containing the paste and the artificial intestinal fluid is placed in a constant-temperature incubator at 37° C., sealed and stored for 3 days; then solution in the beaker is carefully filtered, remaining paste and the beaker are placed in an oven at 60° C. for 18h and taken out, a weight M2 at this time is weighed, and a corrosion resistance rate of the paste is {1-(M1-M2)/10}*100%;

The above test was repeated for 3 times, and an average value was recorded as corrosion resistance of the paste.

Results of material performance test are shown in Table 1:

TABLE 1

| | Corrosion |
|---|---|
| | Resistance |
| Items | Rate |
| Embodiment 1 | 91% |
| Embodiment 2 | 93% |
| Embodiment 3 | 94% |
| Comparative | 56% |
| Embodiment 1 | |
| Comparative | 76% |
| Embodiment 2 | |
| Comparative | 94% |
| Embodiment 3 | |
| Comparative | 9% |
| Embodiment 4 | |

Results are analyzed as follows. In Embodiments 1-3 and Comparative

Embodiment 3, due to addition of a certain amount of the iota-carrageenan and the sericite powder, test results of the paste's corrosion resistant rate show that its corrosion resistance rate is more than 90%, which indicates effect of corrosion resistance and no ease of disintegration in the intestinal fluid. There is no iota-carrageenan in Comparative Embodiment 1, and the paste is easy to disintegrate when heated, with a low corrosion resistance rate; In Comparative Example 2, there was no sericite powder, and without synergistic effect with iota-carrageenan, the paste partially disintegrated, with a relatively low corrosion resistance. In Comparative Example 4, there was no iota-carrageenan and sericite powder, and without carrageenan and sericite powder, the paste would all be disintegrated by the intestinal fluid, and an original shape cannot be maintained, with a poor corrosion resistance rate.

Experiment 2: Clinical Trial Results

A total of 125 patients with faecal dermatitis who insisted on using the paste according to the present disclosure were recruited. In a first group, 29 cases were treated with the leak-proof paste prepared in the formula of the present disclosure in Embodiment 1; In a second group, 28 cases were treated with the leak-proof paste prepared in the formula of Embodiment 2; In a third group, 28 cases were treated with the leak-proof paste prepared in the formula of Embodiment 3; In a fourth group, 24 cases were treated with the leak-proof paste prepared in the formula of Comparative Example 3; and in a fifth group, 16 cases were treated with the leak-proof paste prepared in the formula of Comparative Example 4.

One month after the test:

Cases with the paste disintegrated within 3 days: 1 case in the first group; 2 cases in the second group; 1 case in the third group; 1 case in the fourth group; 13 cases in the fifth group, with a total effective rate of 95.3%.

Case without alleviated inflammation of fecal dermatitis: 0 case in the first group; 1 case in the second group; 0 case in the third group; 18 cases in the fourth group; 14 cases in the fifth group, with a total effective rate of 98.8%.

Causes of main differences are analyzed as follows. The iota-carrageenan can form a strong gel network structure with calcium ions in the stoma excreta. The gel network structure is elastic and insoluble in water and its strength increases with increase of the calcium ions in solution, so the paste can self-regulate its strength with amount of the stoma excreta. In addition, the sericite has unique volume expansion effect and a two-dimensional sheet configuration with a high aspect ratio, excellent reinforcement effect is exhibited in rubber products, so it can strengthen the paste jointly with the iota-carrageenan. In addition, the sericite powder presents acid and alkali corrosion resistance, and the iota-carrageenan and the sericite powder cooperates to achieve high paste strength and good corrosion resistance. According to results of Embodiments 1-3 and Comparative Embodiments 1-2 and 4, the iota-carrageenan and the sericite have synergistic effect on the corrosion resistance.

The Lithospermum Root contains shikonin, which has good effect of eliminating putridity and engendering flesh. At the same time, the Lithospermum Root can inhibit growth activity of Candida albicans, and protect and promote healing of wounds at the same time. The Angelica sinensis can promote blood circulation, nourish blood and promote granulation. The sesame oil has efficacy of detoxifying and promoting granulation. It can be seen from inflammatory situations of fecal dermatitis of users in Embodiments 1-3 and Comparative Embodiment 3 that Combination of these three medicines presents remarkable astringent effect in treating skin diseases, so it can achieve astringent effect for skin complications and prevent sores.

The basic principles, main features and advantages of the present disclosure are shown and described in the above. It should be understood by those skilled in the industry that the above embodiments do not limit the present disclosure in any form, and all technical solutions obtained by equivalent substitution or equivalent transformation fall within the protection scope of the present disclosure.

What is claimed is:

1. A corrosion-resistant and leak-proof stoma paste with self-regulation of strength, wherein the paste's formula comprises 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight.

2. The corrosion-resistant and leak-proof stoma paste with self-regulation of strength according to claim 1, wherein the formula further comprises 5-15 parts of the stoma skin complication medicine by weight.

3. The corrosion-resistant and leak-proof stoma paste with self-regulation of strength according to claim 2, wherein the stoma skin complication medicine comprises 1-3 parts of Lithospermum Root, 1-3 parts of Angelica sinensis and 3-9 parts of sesame oil, by weight.

4. The corrosion-resistant and leak-proof stoma paste with self-regulation of strength according to claim 3, wherein a weight ratio of the Lithospermum Root to the sesame oil is 1: 3.

5. The corrosion-resistant and leak-proof stoma paste with self-regulation of strength according to claim 1, wherein the liquid rubber with the molecular weight ranging from 2000 to 5000 is one or more of polyisobutylene, liquid polyisoprene and liquid styrene-butadiene rubber with a molecular weight ranging from 2000 to 5000.

6. The corrosion-resistant and leak-proof stoma paste with self-regulation of strength according to claim 1, wherein the mineral oil is one or more of naphthenic oil, white oil and paraffin oil.

7. The corrosion-resistant and leak-proof stoma paste with self-regulation of strength according to claim 1, wherein the plant polysaccharide is one or more of tamarind seed polysaccharide, seaweed polysaccharide, chitosan oligosaccharide, tremella polysaccharide, hawthorn polysaccharide and oat μ- glucan.

8. A preparation method of a corrosion-resistant and leak-proof stoma paste with self-regulation of strength, comprising:
Step 1, preparing materials according to a formula;
the formula comprising 4-40 parts of liquid rubber with a molecular weight ranging from 2000 to 5000, 8-18 parts of mineral oil, 14-34 parts of iota-carrageenan, 10-20 parts of plant polysaccharide and 15-20 parts of sericite, by weight; and
Step 2, evenly mixing the liquid rubber with the molecular weight ranging from 2000 to 5000, the mineral oil, the iota-carrageenan, the plant polysaccharide and the sericite, and stirring to obtain a mixed paste, which is packaged to obtain a finished product.

9. The preparation method of the corrosion-resistant and leak-proof stoma paste with self-regulation of strength according to claim 8, wherein the formula further comprises 1-3 parts of Lithospermum Root, 1-3 parts of Angelica sinensis and 3-9 parts of sesame oil, by weight;
wherein heating the sesame oil to 90 ° C. and then adding the weighed Lithospermum Root and the Angelica sinensis, decocting for lh, and filtering to obtain medicinal oil;
wherein add the obtained medicinal oil while evenly mixing the liquid rubber with the molecular weight ranging from 2000 to 5000, the mineral oil, the iota-carrageenan, the plant polysaccharide and the sericite.

10. The preparation method of the corrosion-resistant and leak-proof stoma paste with self-regulation of strength according to claim 9, wherein a double planetary stirrer is adopted for stirring, with a rotating speed of 80-180 r/min.

11. The preparation method of the corrosion-resistant and leak-proof stoma paste with self-regulation of strength according to claim 9, wherein a weight ratio of the Lithospermum Root to the sesame oil is 1: 3.

12. The preparation method of the corrosion-resistant and leak-proof stoma paste with self-regulation of strength according to claim 9, wherein the liquid rubber with the molecular weight ranging from 2000 to 5000 is one or more of polyisobutylene, liquid polyisoprene and liquid styrene-butadiene rubber with a molecular weight ranging from 2000 to 5000.

13. The preparation method of the corrosion-resistant and leak-proof stoma paste with self-regulation of strength according to claim 9, wherein the mineral oil is one or more of naphthenic oil, white oil and paraffin oil.

14. The preparation method of the corrosion-resistant and leak-proof stoma paste with self-regulation of strength according to claim 9, wherein the plant polysaccharide is one or more of tamarind seed polysaccharide, seaweed polysaccharide, chitosan oligosaccharide, tremella polysaccharide, hawthorn polysaccharide and oat μ- glucan.
